(19) United States
(12) Patent Application Publication
(10) Pub. No.: US 2003/0228684 A1
(43) Pub. Date: Dec. 11, 2003

(54) Photobioreactor

(76) Inventors: Ian Michael Burbidge, Surrey (GB); Jonathan Desmond Harper, Matlock (GB)

Correspondence Address: HESLIN ROTHENBERG FARLEY & MESITI PC, 5 COLUMBIA CIRCLE, ALBANY, NY, 12203, US

(21) Appl. No.: 09/795,578

(22) Filed: Feb. 28, 2001

(30) Foreign Application Priority Data
Aug. 9, 1999 (WO) WO 00/12673
Aug. 28, 1998 (GB) 9818931.9
Jan. 26, 1999 (GB) 9901709.7

(51) Int. Cl. C12M 1/00

(52) U.S. Cl. 435/292.1

(57) ABSTRACT

There is provided a photobioreactor comprising an upstanding core structure; a plurality of substantially transparent tubes supportable by the core structure; flow means for causing a synthesis mixture to flow through each of the transparent tubes; and withdrawal means for withdrawing a biomass synthesis product from the mixture. The plurality of transparent tubes is helically wound in parallel. There is also provided the use of the photobioreactor in the production of biomass from a synthesis mixture comprising living plant matter together with essential nutrients for growth of the plant matter.

### CROSS-REFERENCE TO RELATED APPLICATIONS

This application is a continuation application of International application WO 00/12673, filed Aug. 9, 1999.

### FIELD OF THE INVENTION The present invention relates to a photobioreactor suitable for use in methods of biomass production.

### BACKGROUND TO THE INVENTION

The commercial potential of producing biomass products by photosynthesis techniques using simple plant matter, such as algae, blue green bacteria and seaweed, has been recognized for some time. Such techniques seek to harness the ability of simple single cell organisms, such as blue green algae, to utilize sunlight, carbon dioxide and optionally various inorganic constituents to produce more complex biomass products.

Methods involving open channel cultivation of algae have been attempted to produce a biomass product for animal or human consumption. Such open channel methods have proved impracticable for production of pure high grade products because of problems such as invasion by hostile species, contamination by external pollutants, low yield resulting from escape of carbon dioxide to the atmosphere and inefficient use of light to illuminate only the top portion of the biomass.

Methods involving cultivation under more closed conditions have also been suggested. GB-A-2118572, for example, describes a photobioreactor which comprises a plurality of straight transparent tubes arranged substantially horizontally one above the other in a vertical stack. The tubes are connected together in series and a synthesis mixture is caused to flow downwardly through the tubes in a turbulent manner. The tubes are illuminated by natural light whilst the synthesis mixture is passed through them. A biomass synthesis product is withdrawn from the mixture.

EP-A-0239272 describes a photobioreactor comprising an upstanding core structure of substantially cylindrical form. A single, substantially transparent tube is wound helically around the outside of the core structure so that, in use, the exterior of the tube is exposed to natural light. Means are provided for causing a synthesis mixture comprising living plant matter together with essential nutrients for growth of the plant matter to flow under turbulent conditions through the transparent tube. Means are also provided for withdrawing a biomass synthesis product from the mixture. Light is encouraged to penetrate into the tube in the region of contact between the tube and the core structure. Also described are biomass production systems relying on the use of a plurality of single tube winding photobioreactors connected such as to provide parallel flows of the synthesis mixture.

The Applicants have now found that various problems are associated with a photobioreactor having a single tube wound helically around a support structure. In particular, it has been found that for a tube of constant diameter the maximum achievable slope of the helical winding is limited by the outside diameter presented by the supporting structure to the helical tube. As the diameter presented by the supporting structure increases, the maximum achievable slope of the helix decreases. This in turn gives rise to related operating problems. One such problem is the trapping of air or gasses in sags in the tube, which occur more commonly when the slope of the helix is lower. Such trapping of air or gasses can lead to problems in flow of suspended biomass though the tube and also to fouling of the tube, which in turn gives rise to other problems. Another related operating problem is the difficulty in draining down and emptying the tubular coils when the rise in the winding is low, which leads to increased drain down times and also to the need for additional liquids to flush down or sterilize the tubes.

The Applicants have now found that the above described problems associated with a photobioreactor having a single tube wound helically around a support structure can be ameliorated if a plurality of tube windings in a parallel winding arrangement are instead employed.

### SUMMARY OF THE INVENTION

According to one aspect of the present invention there is provided a photobioreactor comprising an upstanding core structure; a plurality of substantially transparent tubes supportable by the core structure; flow means for causing a synthesis mixture to flow through each of the transparent tubes; and withdrawal means for withdrawing a biomass synthesis product from the mixture; wherein said plurality of transparent tubes are helically wound in parallel.

Thus, there is provided a photobioreactor comprising an upstanding core structure; a first substantially transparent tube supportable by the core structure; flow means for causing a synthesis mixture to flow through said first tube; and withdrawal means for withdrawing a biomass synthesis product from the mixture, wherein the first tube is helically wound. One or more additional substantially transparent tubes are additionally provided, each helically wound in parallel to said first tube, and each in communication with the flow means and withdrawal means.

The tubes are helically wound in parallel to each other. The number of tubes is chosen to give a suitable incline in the helical winding. Preferably, from two to ten, most preferably three to five tubes are helically wound in parallel.

Preferably, the support structure is of substantially cylindrical form. It will, however, be appreciated that the support structure is not necessarily truly cylindrical and may, for example, be in the form of a truncated cone. Such a shape can be efficient for light utilization in tropical countries where the sun shines vertically downwards, the conical structure minimizing shadow formation. The support structure may provide a continuous outer surface and be formed for example of hollow concrete sections. Alternatively, the support structure may be of openwork construction or of metal mesh construction.

Preferably, the tube material is polyvinyl chloride, which has excellent light transmission properties and low cost. It also has the valuable advantage of being resistant to attack by the biomass medium. Other plastic materials such as methyl methacrylate or transparent PTFE can be used or even non-plastic materials such as glass if capable of withstanding the conditions of use. If desired for reasons of strength, the tubing may have a reinforcing outer coating, for example of clear resin. This use of such coatings is advisable if the biomass production is to be carried out under considerable pressure.

Preferably, the plurality of tubes is wound helically on the exterior of the support structure. Suitably the tubing is wound at an angle to the horizontal of, for example, 3°. Light is preferably encouraged to penetrate into the tubes in the regions of contact between the tubes and the support structure.

Preferably, the support structure is hollow and comprises a wall of cylindrical form, said having openings provided therein to permit light to pass through the openings and so penetrate into the tube. Preferably, an inlet end of each of said plurality of tubes is connectable to a common inlet manifold and an outlet end of each of said plurality of tubes is connectable to a common outlet manifold.

It is preferred that the reactor comprises a header tank mounted above the tubes and which provides a head of liquid to assist the synthesis mixture to be forced through the tubes. Typically at least an inlet end of each of the tubes is in communication with the header tank, and more usually both the inlet end and an outlet end of each of the tubes are in communication with the header tank so as to form a closed loop from which liquid can be drained, or to which more liquid can be added, as circumstances dictate.

In order to simplify the pipework, an inlet end of each of the plurality of tubes can be connectable to a common inlet manifold and an outlet end of each of the plurality of tubes can be connectable to a common outlet manifold.

Preferably, the flow means comprises an air-lift system, in which the common inlet manifold is in communication with a header tank and a optionally a drain port and the common outlet manifold is in communication with said header tank and a source of air. Alternatively, or additionally, the flow means can comprise a pumping means.

Where an air-lift system is employed, the common outlet manifold typically comprises a down pipe linked to the air lift system. The air-lift system can, for example, comprise a riser pipe linked to the header tank and having a source of air at the lower end thereof. The source of air can be an an air diffuser. The down pipe and riser pipe are preferably connected by connecting pipe means, for example a substantially U-shaped connecting pipe means (e.g. a U-bend). In order to assist assembly and dismantling of the pipework system, the connecting pipe means can comprise a pipe coupling allowing disconnection of the down pipe and riser pipe.

An air vent pipe is preferably provided between the common outlet manifold and the header tank. An advantage of the air vent pipe is that it helps prevent the development of an air block in the manifold which would otherwise impede or prevent movement of the synthesis mixture through the reactor.

The header tank preferably has a water inlet for introducing water and optionally nutrients. Thus as product biomass is drawn off, the system can be replenished by introducing water and/or nutrients through the water inlet. In order to prevent contamination of the synthesis mixture with potentially harmful or undesirable organisms, such as pathogenic organisms, the water inlet is preferably connected to a water supply line having a filter (e.g. a sterilising filter) at an upstream location thereof. The sterilising filter can be, for example a 0.2 micron filter which is capable of preventing the passage therethrough of bacteria and other microorganisms.

The header tank usually is provided with a product outlet, typically mounted at a location between the top and the bottom of the tank. The product outlet can also serve as an overflow and help maintain the liquid in the header tank at a predefined level.

In order to facilitate drainage of the system and subsequent cleaning and sterilising, one or both of the common inlet manifold and common outlet manifold can communicate with a drainage port or ports. One drainage port can be located, for example, in a connecting pipe means (e.g. a U-bend) linking the outlet manifold and riser tube. Another drainage port can be located at the lower end of the inlet manifold.

It is preferred that the drainage ports are arranged so as to leave substantially no dead space within the pipework within which biomass can aggregate. For this reason, it is preferred that the drainage ports extend laterally from the manifolds or other pipework, rather than downwardly.

The system can be cleaned and disinfected or sterilised between growth runs, e.g. at intervals such as six monthly intervals, typically with a suitable chemical sterilising agent such as bleach. Sterilisation can be effected by pumping a suitable sterilising solution through the pipe work and this will enable most surfaces of the pipework to be effectively treated. However, because of air present in the upper end of the header tank, the undersurfaces of the top of the header tank can be difficult or impossible to clean and sterilise effectively merely by pumping sterilising fluid around the system. Therefore, according to a preferred embodiment of the invention, the header tank is provided with a glove port to enable manual cleaning and sterilising without opening the header tank to the atmosphere.

The photobioreactor is preferably provided with monitoring means for monitoring conditions within or about the system. Such monitoring means can include temperature sensors, pH sensors, light meters by way of example. Thus, for example, a temperature sensor can be provided at or near an outlet of one or more of the tubes. In one embodiment, an outlet end of each of the plurality of tubes is connectable to a common outlet manifold, and the temperature sensor is connected to the common outlet manifold.

The monitoring means preferably includes a controller linked to one or more monitoring instruments, for example of the aforesaid type. The controller in turn can be operatively linked to means for varying the system conditions. For example, the controller can be operatively connected to means for controlling any one or more of:

(i) nutrient concentration;

(ii) O₂/CO₂ concentration; and

(iii) temperature.

In one embodiment, the reactor is provided with both a temperature sensor and a pH probe, the temperature sensor and pH probe being linked to a controller, which controller is operatively connected to means for varying the physical environment or the composition of the synthesis mixture.

It is preferred that the temperature sensor and/or controller is or are operatively linked to means for regulating the temperature of the synthesis mixture. In particular, it is preferred that cooling means are provided for cooling the synthesis mixture. The cooling means can comprise an irrigation system for directing cooling fluid (e.g. water) over the tubes. Such an irrigation system can comprise a perforated cooling ring mounted about the upper end of the core and surrounding the tubes, the cooling ring having a supply of water connected thereto. It is preferred that a collection trough is provided at the lower end of the core for collecting the cooling water for recycling. This is particularly preferred when the cooling water is demineralised water.

Preferably, the photobioreactor additionally comprises reflecting means located between the exterior of the support structure and the tubes. If, however, the core structure is of sufficiently open construction, such light reflecting means may not be needed, as sufficient light will penetrate to the underside of the tubing.

The dimensions of the photobioreactor will vary in a manner dependent on the biomass production being carried out. Preferably, the method and apparatus are adapted for continuous production with means being provided for recycle of the synthesis mixture.

The photobioreactor herein may be used in isolation, or alternatively a number of the photobioreactors may be used in parallel or series operation. In one aspect, a plurality of the photobioreactors herein is arranged for parallel flow, wherein each photobioreactor shares a common core support structure.

According to a method aspect of the present invention, there is provided the use of a photobioreactor as described above in the production of biomass from a synthesis mixture comprising living plant matter together with essential nutrients for growth of the plant matter.

Preferably, the synthesis mixture is caused to flow under turbulent conditions through the tube. The use of turbulent conditions enables long running periods before the need for cleaning, so that shut-down periods are kept to a minimum.

Preferably, the synthesis mixture is caused to flow upwardly along the tube. Light can be encouraged to penetrate into the tube in the region of contact between the tube and the support structure.

Preferably, the plant matter comprises algae and/or the essential nutrients for growth comprise carbon dioxide and a source of nitrogen. Ammonia gas may be used as the, or as one of, the nitrogen sources. The use of controlled ammonia injection has been found to be beneficial in minimizing the growth of unwanted microscopic species, such as bacteria, amoebae and rotifers. It is believed that the presence of ammonium salts and ammonium ions inhibits such growth, while acting as a nutrient source for the growth of plant material such as Spirulina (blue green algae).

The nutrients for the synthesis may be provided at least in part by waste effluents such as those from sugar plants or petroleum refinery wastes or other high BOD carbohydrate wastes, the wastes thus being purified in the process, so that the biomass produced is a valuable byproduct of the effluent treatment process.

The method may be carried out under aerobic or anaerobic conditions. Thus, carbon dioxide or air may be supplied to the tube, or other gases, such as oxygen or air/oxygen mixtures, may be employed, depending on the synthesis product desired. Some plant synthesis reactions proceed anaerobically, in which case no such gaseous input is required.

The fact that some biomass synthesis reactions proceed aerobically while some proceed anaerobically can be utilized by providing two or more reactors, as described above, operating in series, a first reactor (or bank of reactors) being used to carry out an anaerobic reaction which leads to evolution of gas, such as carbon dioxide, which, after separation of the first product biomass, is used in a second reactor for an aerobic reaction utilizing the gas.

Preferably, light is encouraged to penetrate into the tube in the region of contact between the tubes and the support structure. The means to encourage light penetration may comprise providing the tube and/or the core with light reflecting means adjacent the area of contact between the tube and the core structure. The light reflecting means is suitably provided by interposition of a material, such as aluminium foil, between the core structure and wound tube. As an alternative, the core structure may be painted white and/or provided with a reflective surface, for example, of small glass balls known as balotini. Alternatively, or in addition, the core may be of sufficiently openwork construction to allow sufficient light to penetrate to the underside of the tubing. To assist light penetration, reflecting means, such as mirrors, may be positioned adjacent the top of the core structure. Alternatively, sufficient illumination within the core may be provided by the inclusion of some form of artificial light source within the hollow centre of the core, such as vertical fluorescent tubes. Such additional illumination may be employed continuously or only when necessary, for example, at night or in very gloomy conditions. Such additional lighting may be set to give flickering illumination to maximize light usage.

### BRIEF DESCRIPTION OF THE DRAWINGS

The invention will now be described by way of example with reference to the accompanying drawings wherein:

FIG. 1 is a diagrammatic view of a photobioreactor in accordance with the invention;

FIG. 2 is a diagrammatic view of an alternative photobioreactor in accordance with the invention:

FIG. 3 is a diagrammatic view of an embodiment similar to that of FIG. 1 but illustrating further features;

FIG. 4 is a diagrammatic view of the inlet and outlet manifolds and air-lift system of the embodiments of FIGS. 1 and 3;

FIG. 5 is an enlarged diagrammatic view illustrating a means of mounting the coiled tubes on the core structure;

FIG. 6 is a view along line A-A of FIG. 5; and

FIG. 7 is a sectional elevation of part of an inlet or outlet manifold.

### DETAILED DESCRIPTION OF THE DRAWINGS

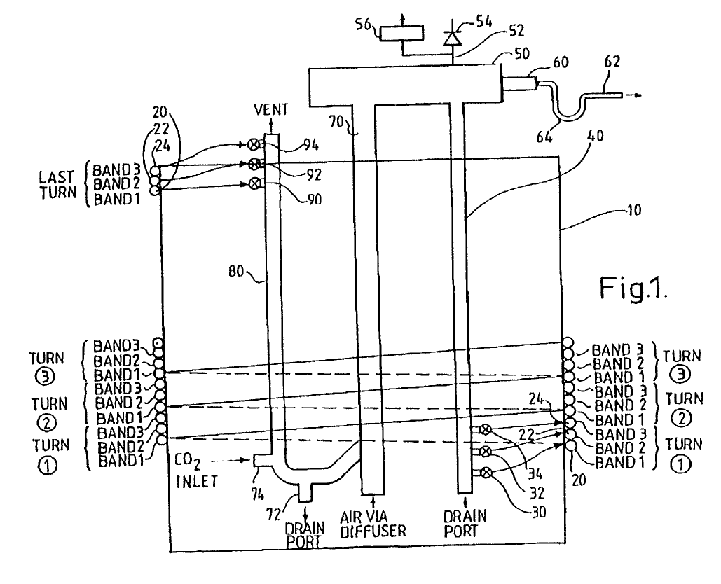

The photobioreactor shown in FIG. 1 comprises a core support structure 10, which is upstanding and substantially cylindrical. Wound helically around the support structure are three (band 1, band 2, band 3) substantially transparent tubes 20, 22, 24. Each of the tubes is wound in parallel fashion to each of the other tubes. Pegs (not shown in FIG. 1) may project from the core support structure 10 to support the tubes 20, 22, 24 and prevent slippage of the windings. In use, synthesis mixture is transported through each of the tubes, generally in an upward direction.

The lower end of the core support structure 10 is mounted in the ground and the lower inlet end of each of the tubes 20, 22, 24 is connected through valves 30, 32, 34 to a downpipe 40, which forms a common inlet manifold. The upper end of the downpipe 40 is in communication with a header tank 50 and the lower end of the downpipe feeds into a drain port (not shown).

The header tank 50 is provided with air outlet 52, wherein the air outlet 52 connects with non-return valve 54 and filter 56. The header tank 50 can contain any suitable means (not shown) to enable a product stream to be withdrawn on line 60, which connects with pipe 62 having trap (or air lock) 64. For example, more concentrated product rising towards the top of the mixture in the launder may be withdrawn by means of a weir device. Alternatively other separation means, such as a hydrocyclone, can replace the header tank 50. Line 60 is shown as extending from the side of header tank 50; however it may equally well extend down the centre of core structure 10 so that product is withdrawn at the base of the structure. The header tank can also contain a purge system to remove excess air and recover the oxygen produced.

The header tank is also connected to riser pipe 70. Air is fed from a diffuser into the lower end of the riser pipe 70. The riser pipe 70 is also provided with a drain outlet 72 and, in this embodiment, a carbon dioxide inlet 74. It will be appreciated however that the carbon dioxide inlet can be positioned at other locations on the pipework if desired. A return manifold 80 also connects with the riser pipe 70. The upper end of the return manifold 80 is connected through valves 90, 92, 94 to the upper outlet ends of the tubes 20, 22, 24.

The product stream on line 60 may pass to any suitable ancillary equipment for treating and/or extracting desired products from the biomass. It is particularly useful to pass the biomass through a solids/liquid or liquid/liquid contactor in cocurrent or countercurrent to a stream of a suitable immiscible extractant. A series of products may be extracted by contacting in a series of contactors with, if necessary, recycle of the raffinate phases between contactors. A suitable extractor is the bucket type contactor known as the Graesser contactor and described in GB patent specification No. 1,145,894.

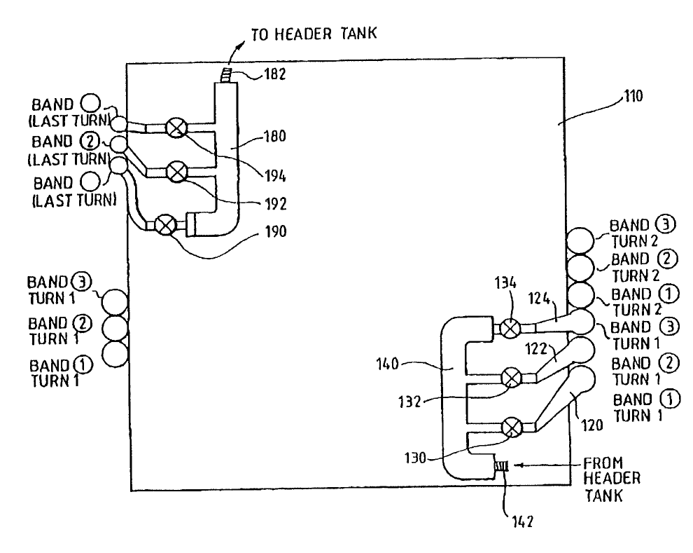

The alternative photobioreactor shown schematically in FIG. 2. also comprises a core support structure 110, which is upstanding and substantially cylindrical. Wound helically around the support structure are three (band 1, band 2, band 3) substantially transparent tubes 120, 122, 124. Each of the tubes is wound in parallel fashion to each of the other tubes. Pegs (not shown) may project from the core support structure 110 to support the tubes 120, 122, 124 and prevent slippage of the windings. In use, synthesis mixture is transported through each of the tubes, generally in an upward direction.

The lower end of the core support structure 110 is mounted in the ground and the lower inlet end of each of the tubes 120, 122, 124 is connected through valve arrangements 130, 132, 134 to a common inlet manifold 140. In use, synthesis mixture is pumped (pumping means not shown) from a header tank (not shown) to the inlet 142 of the common inlet manifold 140 and thence in parallel through the tubes 120, 122, 124. The upper ends of each of the tubes 120, 122, 124 connect through valves 190, 192, 194 to return manifold 180 having outlet 182, thus allowing for return of the synthesis mixture to the header tank.

The pumping means can contain a diaphragm pump or any other suitable type of pump, which may in turn be connected to supplies of, for example, carbon dioxide and/or air, nutrients and a nitrogen source, such as that provided by ammonia, ammonium salts, urea, compound fertilizers etc.

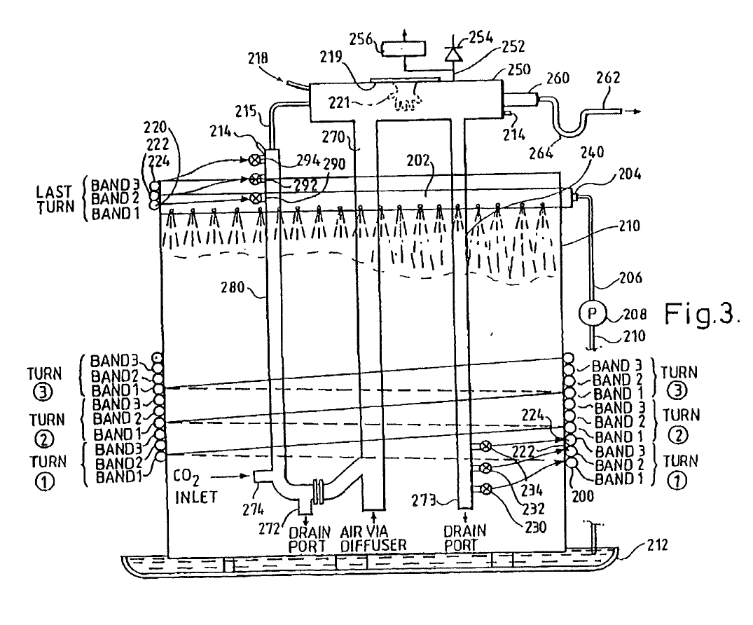

FIG. 3 illustrates schematically a bioreactor of the same general design as the embodiment of FIG. 1 but with several additional features highlighted. In FIG. 3, features corresponding to features found in the reactor of FIG. 1 share the same final two reference numbers but in FIG. 3, the reference numbers are prefixed by the number “2”.

As can be seen in FIG. 3, the bioreactor is provided with a cooling ring which comprises a length of perforated tubing encircling the upper end of the helix of tubes 220, 222, 240. The cooling ring 202 is linked via a T-piece 204 to a tube 206 and thence via a pump 208 and a further length of tube 210 to a collection tray 212.

Set into a port at the upper end of the outlet manifold is a temperature probe 214 linked to a controller (not shown). The controller in turn is linked to pump 208. The temperature probe is located at the top of the helix since the water in the tubes, having had maximum exposure time to sunlight as it reaches this point, will be at its hottest. If the temperature sensor indicates that the temperature is above a certain predetermined threshold value (e.g. the water has reached a temperature which is detrimental to the growing cells within the tubes), the controller actuates the pump to initiate a flow of water to the cooling ring 202 whereupon it can cascade down the outside of the helical coil over the surface of the tubes to cool the synthesis mixture. Since the water is preferably demineralised or deionised, so as to prevent limescale and other mineral build up on the tubes, it is preferred to recycle the water and this is achieved by collecting the water in collection tray 212 and pumping it back to the cooling ring 202 through pipes 206, 210. The level of water in the collection tray can be replenished automatically via a float valve controlled inlet (not shown) in the wall of the tray.

In addition to the temperature probe, the controller can also be linked to other probes which measure other chemical and physical conditions within the tubes. For example, a pH probe 214 can be provided in the wall of the header tank for measuring the relative acidity or alkalinity of the synthesis mixture within the tubes. If the pH departs from an optimal value for the organisms being cultured in the reactor, the controller can be programmed to effect introduction of a pH adjusting substance through a suitably located port. For example, in the case of algae, where the pH of the synthesis mixture increases with algal growth, CO₂ can be introduced to restore the pH to a more acidic value appropriate for the algae.

A further example of a monitoring probe is a light meter, and the controller can be connected to the water/nutrient inlet port 218 in the header tank to control (e.g. via a solenoid valve) the flow of water and nutrients into the header tank. Thus, for example, in bright sunlight, where faster growth of the organisms will occur, nutrient can be fed into the header tank at a faster rate than when the light is relatively poor and growth is slower.

In order to prevent an airlock from developing in the upper end of the outlet manifold, a vent pipe 215 is mounted in a port in the outlet manifold and connects with the header tank. Thus any air present in the system can escape to the header tank, from where it can be exhausted to atmosphere throught the air outlet 252.

It is preferred that the photobioreactor is operated under conditions which prevent contamination of the cells in the synthesis mixture with pathogens or other organisms. Therefore, the reactor is provided with several features which assist in eliminating or keeping out such unwanted organisms. Prior to using the reactor, the pipe work is thoroughly rinsed with a suitable biocidal material such as a bleach which can either be moved around the system using the air-lift facility or can be pumped using an auxiliary pump (not shown). In order to allow synthesis mixture to to be drained from the system and to allow subsequent flushing with biocidal solutions and rinsing water, drain ports 272 and 273 are provided at the lower ends of the down pipe 240 and riser pipe 270 respectively. By pumping biocidal solution around the system, most of the surfaces within the pipework and header tank can be disnfected or sterilised. However, it is difficult to sterilise the under surfaces of the top of the header tank and, therefore, a glove port 219 is provided in which a tough biocide-resistant waterproof glove can be mounted. The glove can then be used to splash cleaning fluid from the header tank onto the undersurface of the top of the tank, or to wipe down otherwise inaccessible surfaces within the tank without the need to open up the tank.

In order to sterilise the system, it is most preferred to sterilise each component of the synthesis mixture before introduction into the reactor, so far as is possible. Thus, for example, water/nutrient solution entering inlet 218 is first filtered to remove macroscopic contaminants and is then subjected to sterile filtration through a 0.2 micron filter which will remove organisms down to and including organisms the size of bacteria. Additionally, the water/nutrient solution can be passed by or through an ultraviolet sterilising device or pasteurising device en route to the header tank.

Sterilising filters and non-return valves are also fitted to the air inlets in the header tank in order to prevent airborne contamination with pathogens or other unwanted microbial species.

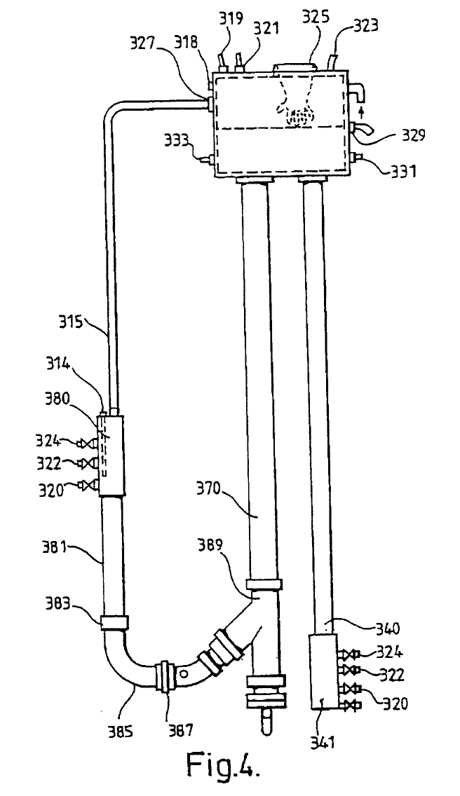

The layout of a typical pipework arrangement is shown more clearly in FIG. 4. Thus, as with the embodiment of FIGS. 1 and 3, three pipes 320, 322 and 324 are helically wound in parallel and are connected at their lower ends to an inlet manifold 340 and at their upper ends to an outlet manifold body 380. Outlet manifold 380 has a down pipe portion 381 extending downwardly therefrom, the downpipe portion 381 being linked by means of coupling 383 to a U-bend 385 which in turn is connected at junction 389 to riser pipe 370. The U-bend 385 contains a coupling 387 (for example a screw collar or a flanged coupling) enabling the pipe work to be disconnected, or to assist assembly. Also contained within the U-bend is drainage valve 391.

At the lower end of the riser pipe 370 is an air diffuser 339 which can be of the form disclosed in our copending application number . . . (internal reference P57627M). Air diffuser 370 injects air into the synthesis mixture thereby causing the mixture to rise up the riser pipe 370 to the header tank.

The upper end of the outlet manifold 380 is provide with two ports, one having mounted therein a temperature probe 314, and the other having mounted therein a vent pipe. The functions of the temperature probe and vent pipe have been described above and need not be repeated here.

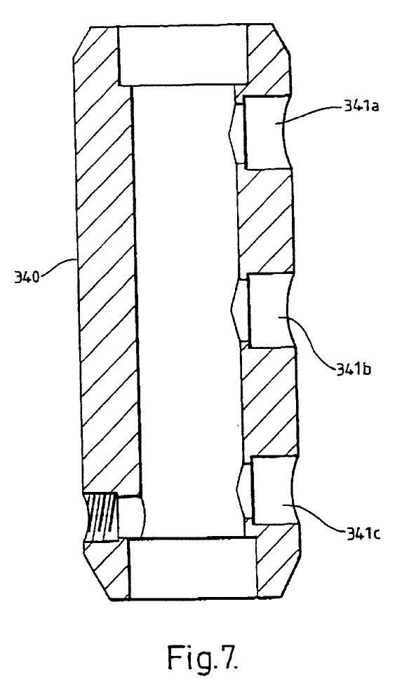

The inlet manifold 340 comprises a thick walled main body portion 341, which is shown in enlarged longitudinal section in FIG. 7. The walls of the manifold body portion are sufficiently thick to enable ports 341a, 341b and 341c to be drilled and the ends of the valved tubes 320, 322, 324 to be mounted and secured therein. Where the down pipe leading to the inlet tubes is of larger diameter and has a thicker wall, the separate thick-walled manifold body portion can be omitted and the ports for the tubes drilled directly into the down pipe. A similar form of construction can also be used for the outlet manifold 381.

The arrangement of the various ports in the header tank can also be seen more clearly in FIG. 4. Thus, the upper part of the header tank has a water/nutrient inlet port 318, an inoculation port 319 (through which starter cultures of, for example, algae, can be introduced), an air outlet port 321 fitted with a non return valve (e.g. a ball valve), an air inlet port 323 connected to a sterile filter to prevent airborne contamination, and a glove port 325. The purpose of the air inlet port is to allow air to bleed back into the system in the event that the air compressor driving the air lift system fails thereby causing a reduction in pressure in the system. The sterile filter prevents contaminants from entering the header tank during any such bleed back. Glove port 325 comprises a cylindrical upstanding rim about which is stretched the opening of a glove formed from a suitably tough waterproof material such as a rubber. The glove is secured to the rim by means of a clamping band 325a.

Located near the top of the header tank is inlet port 327 to which is attached the vent tube 315 from the outlet manifold. On the other side of the header tank, just over halfway up the tank, is the product outlet port/overflow port 329. Mounted in port 329 is an outlet tube through which product mixture can be directed to a harvesting facility or directly to a point of use of the biomass. At the base of the header tank, on opposite sides of the tank, are ports 331 and 333 in which are mounted respectively, a pH probe and a sample run-off tube.

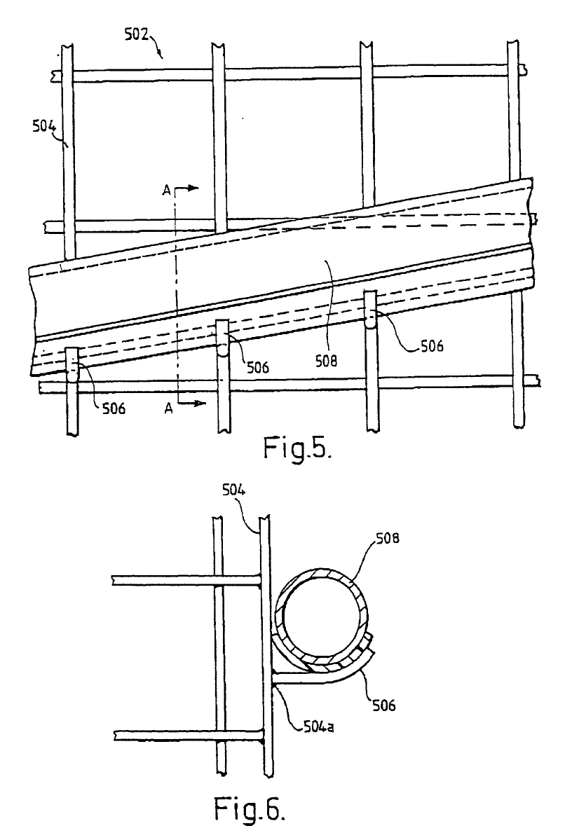

As described in respect of FIG. 1, the core structure can be provided with pegs upon which the tubes are mounted. Examples of suitable peg arrangements are shown in FIGS. 5 and 6. The core structure 502 illustrated in FIG. 5 is formed from a metal mesh 504 which can be galvanised or plastic coated to prevent corrosion. Welded to the mesh at weld points 504a are hook shaped pegs 506 which are lined with a strip of a suitable plastics material such as PVC tubing to prevent abrasion and wear on the reactor tube which is mounted on the peg.

It will be appreciated that the photobioreactors described in detail above are easy to assemble and if wished can be constructed in modular form. Thus the tube can readily be constructed in sections with valves and junctions allowing reaction products to be removed as needed and/or any necessary additional nutrient feed introduced. This is especially useful for rapid reactions, such as certain fermentation reactions.

Although an air lift operation has been described above for providing the motive force necessary to cause flow in the tubing, it may be desirable in some reactions to employ higher flow conditions, for example, where the product cells are of a less delicate nature. In such cases pumps, for example of conventional design, may be used to sustain circulation. If need be a compressed gas venturi jet or a steam jet may be employed. Steam injection is particularly suitable where a certain amount of heat is required for growth. Provided that the organisms to be grown in the reactor are robust enough, the header tank and air lift system can be dispensed with altogether and one or more pumps used instead as the sole means for circulating the synthesis mixture.

Provision may be made for intermediate pumps, and/or air or steam injection so as to control the flow rates through the tubing even for long flow passages. This is particularly valuable when the reaction medium has a tendency to become viscous, for example in certain fermentation processes.

The method and apparatus described above are applicable to a wide range of biomass production processes. It will be appreciated that considerable variation is possible in the nutrients supplied to the bioreactor and the operating conditions.

If desired, the feed system to the reactor can be controlled to introduce small amounts of one or more trace elements such as selenium, cobalt, copper, zinc, gallium and germanium under varying conditions to alter trace element amounts.

The provision of a continuously operable process with recycling of the mixture keeps the consumption of gases and nutrients as low as possible with minimum wastage. Product oxygen can be used in any adjacent chemical plant. Any heat produced can be employed in heat exchange.

1. A photobioreactor comprising an upstanding core structure; a plurality of substantially transparent tubes supportable by the core structure; flow means for causing a synthesis mixture to flow through each of the transparent tubes; and withdrawal means for withdrawing a biomass synthesis product from the mixture; wherein said plurality of transparent tubes are helically wound in parallel.

2. A photobioreactor according to claim 1, wherein the support structure is of substantially cylindrical form.

3. A photobioreactor according to claim 1 wherein the plurality of tubes are wound helically on the exterior of the support structure and light is encouraged to penetrate into the tubes in the regions of contact between the tubes and the support structure.

4. A photobioreactor according to claim 1 wherein the support structure is hollow and comprises a wall of cylindrical form, said having openings provided therein to permit light to pass through the openings and so penetrate into the tube.

5. A photobioreactor according to claim 1 wherein at least an inlet end of each of the tubes is in communication with a header tank.

6. A photobioreactor according to claim 5 wherein both the inlet end and an outlet end of each of the tubes are in communication with the header tank so as to form a loop.

7. A photobioreactor according to claim 1 wherein an inlet end of each of said plurality of tubes is connectable to a common inlet manifold and an outlet end of each of said plurality of tubes is connectable to a common outlet manifold.

8. A photobioreactor according to claim 7, wherein the flow means comprises an airlift system, in which said common inlet manifold is in communication with a header tank and optionally a drain port and said common outlet manifold is in communication with said header tank and a source of air.

9. A photobioreactor according to claim 8 wherein the common outlet manifold comprises a down pipe linked to the air lift system.

10. A photobioreactor according to claim 9 wherein the air lift system comprises a riser pipe linked to the header tank and having a source of air at the lower end thereof.

11. A photobioreactor according to claim 10 wherein the source of air is an air diffuser.

12. A photobioreactor according to claim 10 wherein the down pipe and riser pipe are connected by connecting pipe means, for example a substantially U-shaped connecting pipe means.

13. A photobioreactor according to claim 12 wherein the connecting pipe means comprises a pipe coupling allowing disconnection of the down pipe and riser pipe.

14. A photobioreactor according to claim 8 wherein an air vent pipe is provided between the common outlet manifold and the header tank.

15. A photobioreactor according to claim 5 wherein the header tank has a water inlet for introducing water and optionally nutrients.

16. A photobioreactor according to claim 16 wherein the water inlet is connected to a water supply line having a filter at an upstream location thereof.

17. A photobioreactor according to claim 5 wherein the header tank has a product outlet.

18. A photobioreactor according to claim 7 wherein one or both of the common inlet manifold and common outlet manifold are in communication with a drainage port or ports.

19. A photobioreactor according to claim 12 wherein the connecting pipe means comprises a drainage port.

20. A photobioreactor according to claim 7 wherein said common inlet manifold is in communication with a drain port.

21. A photobioreactor according to claim 5 wherein the header tank is provided with a glove port, for example in a top surface thereof.

22. A photobioreactor according to claim 1 wherein a temperature sensor is provided at or near an outlet of one or more of the tubes.

23. A photobioreactor according to claim 22 wherein an outlet end of each of the plurality of tubes is connectable to a common outlet manifold, and the temperature sensor is connected to the common outlet manifold.

24. A photobioreactor according to claim 1 which includes a probe for measuring pH.

25. A photobioreactor according to claim 24 wherein the pH probe is mounted in a wall of the header tank.

26. A photobioreactor according to claim 22 having both a temperature sensor and a pH probe, the temperature sensor and pH probe being linked to a controller, which controller is operatively connected to means for varying the physical environment or the composition of the synthesis mixture.

27. A photobioreactor according to claim 26 wherein the controller is operatively connected to means for controlling any one or more parameters selected from:
(i) nutrient concentration;
(ii) O₂/CO₂ concentration; and
(iii) temperature.

28. A photobioreactor according to claim 26 wherein the temperature sensor and/or controller is or are operatively linked to means for regulating the temperature of the synthesis mixture.

29. A photobioreactor according to claim 1 wherein cooling means are provided for cooling the synthesis mixture.

30. A photobioreactor according to claim 29 wherein the cooling means comprises an irrigation system for directing cooling fluid over the tubes.

31. A photobioreactor according to claim 30 wherein the irrigation system comprises a perforated cooling ring mounted about the upper end of the core and surrounding the tubes, the cooling ring having a supply of water connected thereto.

32. A photobioreactor according to claim 31 wherein a collection trough is provided at the lower end of the core for collecting the cooling water for recycling.

33. A photobioreactor according to claim 30 wherein means are provided for demineralising the cooling water.

34. A photobioreactor according to claim 1 wherein said flow means comprises one or more pumps.

35. A photobioreactor according to claim 34 wherein a pump represents the primary flow means.

36. A photobioreactor according to claim 1 additionally comprising reflecting means located between the exterior of the support structure and the tubes.

37. A method for the production of biomass comprising using a photobioreactor according to claim 1 to produce said biomass from a synthesis mixture of living plant matter together with essential nutrients for growth of the plant matter.

38. The method of claim 37, wherein light is encouraged to penetrate into the tube in the region of contact between the tubes and the support structure.

39. The method of claim 37 wherein the plant matter comprises algae, blue green bacteria or seaweed and/or wherein the essential nutrients for growth comprise carbon dioxide and a source of nitrogen, preferably ammonia gas.

40. A photobioreactor comprising an upstanding support structure; a plurality of substantially transparent tubes supportable by the support structure; flow means for causing a synthesis mixture to flow through each of the transparent tubes; and withdrawal means for withdrawing a biomass synthesis product from the mixture; wherein said plurality of transparent tubes are helically wound in parallel.
